(19) United States
(12) Patent Application Publication
(10) Pub. No.: US 2026/0315637 A1
(43) Pub. Date: Oct. 15, 2026

(54) EVALUATION OF A SANITIZATION PROCEDURE FOR BIOPROCESSING EQUIPMENT

(71) Applicant: Cytiva Sweden AB, Uppsala (SE)

(72) Inventors: Kerstin Erickson, Uppsala (SE); Henrik Ingvarsson, Uppsala (SE)

(21) Appl. No.: 19/483,536

(22) PCT Filed: May 21, 2024
(86) PCT No.: PCT/EP2024/063922

§ 371 Date: Nov. 11, 2025

(30) Foreign Application Priority Data
May 24, 2023 (SE) 2350629-8

(51) Int. Cl. A61L 2/28; C12Q 1/22

(52) U.S. Cl. CPC A61L 2/28; C12Q 1/22

(57) ABSTRACT

A procedure 10 for evaluating a sanitization procedure used for sanitizing bioprocessing equipment or for evaluating the hygienic design of bioprocessing equipment is disclosed. The procedure comprises: pre-cleaning 100 the bioprocessing equipment; providing 101 a mixture of test microorganisms comprising two or more different microorganism strains; applying 102 the mixture of test microorganisms in the bioprocessing equipment; waiting for at least 2 hours; sanitizing 104 the bioprocessing equipment; collecting 105 at least one test sample, possibly comprising test microorganisms, from at least one point in the bioprocessing equipment after the sanitization; transferring 106 at least a portion of a collected test sample into/onto a culture medium; incubating 107 the culture medium, allowing possibly present test microorganisms to grow and differentiate in/on the culture medium, and identifying and/or counting the amount of test microorganisms 108 of each microorganism strain grown on/in the culture medium.

### TECHNICAL FIELD

The present disclosure relates to evaluation of a sanitization procedure used for sanitizing bioprocessing equipment or for evaluating the hygienic design of a bioprocessing equipment.

### BACKGROUND ART

Microbial contaminations, i.e. bacteria, yeast and mould, are found in many laboratory and production environments. Growing rapidly to large quantities under favourable conditions, these microorganisms can damage the function and impair the performance of bioprocessing equipment, such as chromatographic equipment and chromatography resins. Additionally, microorganisms can remain as contaminants of the bio-product throughout manufacturing, with batch failure and related costs as a consequence. Consequently, it is important to follow hygienic routines throughout the whole production process for the safety of patients, for compliance with Good Manufacturing Practice (GMP) and to meet strict regulations.

Sanitization, defined as the use of chemical agents to reduce microbial populations, is commonly used to maintain microbial presence in bioprocessing equipment at levels that minimize the risk of batch failures and product contamination. Sanitization can also be used to evaluate the hygienic design of bioprocessing equipment. To evaluate a sanitization procedure used for sanitizing bioprocessing equipment or to evaluate a hygienic design thereof and whether a change in sanitization procedure or hygienic design is needed, is both time consuming and costly.

### SUMMARY OF THE INVENTION

It is an object of the present disclosure to provide a faster and/or an improved procedure for evaluating a sanitization procedure used for sanitizing bioprocessing equipment or for evaluating the hygienic design of bioprocessing equipment.

The invention is defined by the appended independent patent claims. Non-limiting embodiments emerge from the dependent claims, the appended drawings, and the following description.

According to a first aspect, there is provided a procedure for evaluating the effectiveness of a sanitization procedure used for sanitizing bioprocessing equipment or for evaluating the hygienic design of bioprocessing equipment, comprising: pre-cleaning the bioprocessing equipment; providing a mixture of test microorganisms comprising two or more different microorganism strains; applying the mixture of test microorganisms in the bioprocessing equipment; waiting for at least 2 hours, sanitizing the bioprocessing equipment; collecting at least one test sample, possibly comprising test microorganisms, from at least one point in the bioprocessing equipment after the sanitization step; transferring at least a portion of a collected test sample into/onto a culture medium; incubating the culture medium, allowing possibly present test microorganisms to grow and differentiate in/on the culture medium, and identifying and/or counting the amount of test microorganisms of each microorganism strain grown on/in the culture medium. The latter step of identifying and/or counting the amount of test microorganisms of each microorganism strain grown on/in the culture medium may preferably entail separately quantifying the amount of each respective microorganism as it is grown simultaneously together with the other microorganism(s) in the same culture medium (e.g. on the same agar plate).

With bioprocessing equipment is here meant equipment or parts thereof used at any stage of bioprocessing, i.e. in upstream, midstream or downstream processes. Bioprocessing equipment here includes components, assembly of components (modules), complete systems, and complete process lines with several systems. Examples are: chromatography systems, inline conditioning systems, columns and 3D printed components. The described procedure is used for evaluating the effectiveness of a sanitization procedure used for sanitizing bioprocessing equipment or for evaluating the hygienic design of bioprocessing equipment. The procedure gives information whether a change in sanitization procedure or hygienic design is needed.

An effective sanitization of bioprocessing equipment is very important for the safety of patients, to comply with Good Manufacturing Practice (GMP) and regulations. Failure of compliance is associated with high costs for pharmaceutical companies with batch losses and lengthy and resource demanding investigations. Therefore, it is of importance to understand if a chosen sanitization procedure meets the demands such that the product is compliant with GMP and regulations.

Traditionally, a sanitization procedure may be evaluated by challenging the system with one test microorganism at a time, which is then evaluated. If additional test microorganisms are used, these are tested in sequence. The more test microorganisms, the longer the evaluation time. Such sanitization studies are, hence, both time consuming and costly.

The above described procedure, as well as traditional procedures, give information on how the used sanitization procedure performs and whether a change of the hygienic design and/or the sanitization procedure is needed or not. Presence of one or more test microorganism and number thereof in/on the culture medium is an indication that the hygienic design and/or sanitization procedure need to be changed.

Before microorganisms are applied in the system, all parts of the system are pre-cleaned by using for example a detergent and soft brush, followed by treatment with a sanitization agent or by use of autoclavation (if appropriate, considering material properties and/or size). Pre-cleaning/pre-sanitization is performed to mitigate risk of there being microorganisms already in the system when applying the mixture of test microorganisms, which otherwise could affect the results/conclusions drawn.

The amount of each microorganism in the mixture of test microorganisms comprising two or more different microorganism strains applied in the system may be >10³ CFU/ml, or >10⁴ CFU/ml, or >10⁵ CFU/ml or >10⁶ CFU/ml.

The test microorganisms in the mixture should be applied in the bioprocessing equipment with a defined concentration of each microorganism. The test microorganisms are mixed in an isotonic solution, such as 0.9% NaCl, before applied in the system. Preferably, the test microorganisms are present in the mixture in approximately the same concentrations.

The mixture of microorganisms may be applied at one or more positions in the bioprocessing equipment (depending on equipment and size thereof).

When the mixture of test microorganisms has been applied in the bioprocessing equipment, there is a step of waiting for at least 2 hours. The waiting period may be 2-48 hours, at least 4 hours, at least 8 hours at least 12 hours, at least 16 hours, at least 20 hours, at least 24 hours, at least 30 hours, 4-20 hours, or 10-18 hours.

The waiting time chosen may depend on the mixture of test microorganisms used. Different microorganisms sediment, start to form bounds to surfaces of the bioprocessing equipment and eventually form biofilms after different amounts of time have passed.

After the waiting step, the bioprocessing equipment is sanitized with a defined sanitization method.

After sanitization, at least one test sample, possibly comprising test microorganisms, is collected from at least one point, or from 1-70 different points, or from 10-20 different points in the bioprocessing equipment. From each point in the processing equipment 1-70, or 10-20, or 20-70 separate samples may be taken. The samples may then be analysed in parallel. The samples may for example be a liquid volume or a sample taken using contact plates or a swab or tops. Test samples may be collected at several positions in the equipment and analysed in parallel.

At least a portion of a collected test sample is then transferred into/onto a culture medium, the culture medium promoting growth and differentiation of all test microorganism strains in the mixture of test microorganisms applied in the bioprocessing equipment, thereby allowing possibly present test microorganisms to grow and differentiate in/on the same culture medium sample. Also, any unintentional organism in the collected sample, not part of the test mixture, but present in the bioprocessing system, is allowed to grow and differentiate in/on the culture medium.

After cultivation, test microorganisms, as well as any present unintentional organism, are identified and/or the number of test microorganisms of each microorganism strain, or any unintentional organism, counted.

Depending on the acceptance criteria for a specific bioprocessing equipment or acceptance criteria for different microorganisms, the result from the identification and/or counting of microorganisms of different strains may indicate that the sanitization procedure is inadequate and that another/improved sanitization procedure is needed. The result may also indicate if the hygienic design of a bioprocessing equipment is satisfying or if a redesign is needed.

The amount of each test microorganisms in the mixture of microorganisms applied in the bioprocessing equipment may for example be 10⁶ CFU/ml. A criteria for a successful sanitization procedure could then be that after sanitization and after cultivation the number of each test microorganism in a collected sample is less than 100 CFU/ml or less than 10 CFU/ml. If the amount is higher the sanitization procedure does not meet the criteria and another sanitization procedure is needed.

Compared to traditional evaluation of sanitization procedures, the present procedure uses two or more test microorganisms mixed together before application. Collected samples possibly comprising the test microorganisms are then analysed on/in the same culture medium. Thereby, there is a significant reduction in the time compared to if two test microorganisms are sequentially evaluated. The described procedure could, hence, reduce the time needed for sanitization evaluation from three/four months into possibly only one month. More data is generated in a shorter amount of time, which reduces the total project time and optimizes the use of resources in a better way.

The mixture of test microorganisms comprises two or more different microorganism strains that may be selected from Escherichia coli, Pseudomonas aeruginosa, Staphylococcus aureus, Candida albicans, Bacillus subtilis, Aspergillus niger, Pichia pastoris and Saccharomyces cerevisiae.

Such microorganisms are preferably USP (US Pharmacopeia) recommended organisms for use in testing nonsterile products. The chosen microorganism strains (Escherichia coli (gram negative bacteria), Pseudomonas aeruginosa (gram negative bacteria), Staphylococcus aureus (gram positive bacteria), Candida albicans (yeast), Bacillus subtilis (gram positive bacteria), Aspergillus niger (mould), Pichia pastoris (yeast, not USP recommended) and Saccharomyces cerevisiae (yeast, not USP recommended) cover a broad spectrum of microorganisms commonly encountered in bioprocessing industries. Since these microorganisms mutually differ from each other regarding morphology, size and attraction towards different materials, a more robust and realistic test scenario is achieved with regard to hygienic design, thereby mitigating risks for time delays (redesign of product or test method).

The mixture of selected microorganism may comprise 2-4 strains of microorganisms.

If using a mixture of two or three test microorganisms the test microorganisms may for example comprise Escherichia coli, Pseudomonas aeruginosa, and/or Staphylococcus aureus.

If using four test microorganisms in the mixture the microorganisms used may for example be Escherichia coli, Pseudomonas aeruginosa, Staphylococcus aureus and Candida albicans.

The listed test microorganisms are all possible to co-cultivate in/on the same culture medium.

The step of waiting for at least two hours may be performed at a temperature of 15-30 °C.

Alternatively, the temperature used may be lower than 15 °C, SUCH AS 4-14 °C.

The sanitization of the bioprocessing equipment may be made using any one or more of sodium hydroxide, isopropanol, ethanol, peracetic acid, hydrogen peroxide, acetic acid, citric acid, sodium hypochlorite (chlorine), quaternary ammonium compounds (e.g. benzalkonium chloride), iodine-based disinfectants (e.g. povidone-iodine), phenols (e.g. chlorophenol), aldehydes (e.g. formaldehyde and glutaraldehyde), ammonia, nitiric acid, phosphoric acid, sulfuric acid, peroxyacetic acid, potassium permanganate, potassium hydroxide, ammonium chloride, sodium bicarbonate, chlorhexidine and perethylene.

The sanitization agents listed may have various concentrations and be used in various combinations. For example, there could first be one or more acid sanitization steps followed by one or more basic sanitization steps or vice versa. Sanitization, defined as the use of chemical agents to reduce microbial populations, is commonly used for chromatography systems to maintain microbial presence at levels that minimize the risk of contaminating the bioproduct.

Sanitization could also, depending on the bioprocessing equipment and materials used, include gamma radiation.

A test sample may further be collected from at least one point in the bioprocessing equipment after application and/or immediately before the sanitization step, each test sample being transferred into/onto a culture medium, incubated to allow possibly present microorganisms to grow and differentiate, and thereafter each microorganism strain grown on/in the culture medium is identified and/or the number of microorganisms counted.

This procedure is performed to check viability and concentration of each test microorganism in the mixture of microorganisms at different time points. The amount of each test microorganism in a starting mixture of microorganisms may for example be about 10⁶ CFU/mL. After application and/or immediately before sanitization, the viability and concentration of each test microorganism in a collected test sample is analyzed. If the concentration has decreased under a pre-defined minimum amount of test microorganisms after application or just before sanitization (such as below 10⁶ CFU/mL), this indicates that the experimental procedure or set-up is not working and the procedure is interrupted before sanitization, as it will not be possible to evaluate the sanitization process used.

The effectiveness of the hygienic design or the chosen sanitization method can be evaluated if the amount/type of microorganism present before/during sanitization is known. First, the number of the organisms within the system is established and after sanitization the absence of organisms is investigated.

The procedure may comprise a step of filtering the collected test sample before transferring at least a portion of the filtered sample or at least a portion of the filter into/onto a culture medium.

Filtering may be performed with a suitable pour size (for example: 0.45 μm) to capture presence of microorganisms, and thereafter transfer the filters into/onto a suitable culture medium. Filtering may also be performed to remove other components than microorganisms from a liquid test sample.

Incubation of the culture medium, allowing possibly present microorganisms to grow and differentiate, may be made at a temperature of 20-42 °C, during a time-period of 1 to 5 days, at a pH of 6.0-8.0 and an osmotic pressure of 0.05-2.5 M.

The culture medium may be a solid or semi-solid culture medium.

Solid media may contain agar at a concentration of 1.5 to 2.0 % concentration. Semi-solid media may contain agar at a concentration of 0.2-0.5 %.

The culture medium may alternatively be a broth.

The culture medium may comprise a carbon source selected from any one or more of glucose, lactose, sucrose, glycerol, fructose, mannitol, succinate, maltose, xylose and galactose.

The culture medium may comprise a nitrogen source selected from any one or more of peptone, tryptone, yeast extract, ammonium sulphate, and ammonium chloride.

The culture medium may comprise any one or more of iron, manganese, coper, cobalt, molybdenum, nickel, selenium, biotin, folic acid, nicotinic acid, pantothenic acid, pyridoxine, riboflavin, thiamine, vitamin B12, vitamin K, inositol and para-aminobenzoic acid.

### BRIEF DESCRIPTION OF THE DRAWINGS

FIG. 1 schematically illustrates the general steps in a sanitization procedure 10 used for sanitizing bioprocessing equipment or for evaluating the hygienic design of bioprocessing equipment.

FIG. 2 shows an agar plate 20 with three co-cultured microorganisms: 1) Staphylococcus aureus, 2) Escherichia coli and 3) Pseudomonas Aeruginosa.

### DETAILED DESCRIPTION

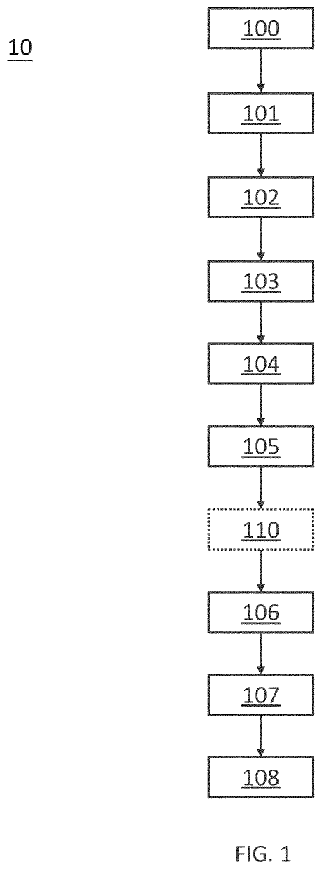

FIG. 1 schematically illustrates a sanitization procedure 10 used for sanitizing bioprocessing equipment or for evaluating the hygienic design of bioprocessing equipment. An effective sanitization is very important to comply with Good Manufacturing Practice (GMP) and regulations. Failure of compliance may be associated with high costs. The present procedure gives information on how the used sanitization process performs and whether a change in sanitization process is needed or not. Compared to traditional sanitization evaluation processes, the procedure described here is faster and cheaper. The procedure can also be used to evaluate the hygienic design of bioprocessing equipment and whether a change in hygienic design is needed.

The procedure comprises a step of pre-cleaning 100 the bioprocessing equipment, using e.g. detergents, autoclavation, antimicrobial agents etc. A mixture of test microorganisms of two or more, such as 2-4, different microorganism strains is provided 101. The microorganism strains may be selected from Escherichia coli, Pseudomonas aeruginosa, Staphylococcus aureus, Candida albicans, Bacillus subtilis, Aspergillus niger, Pichia pastoris andi Saccharomyces cerevisiae. The chosen test microorganisms cover a broad spectrum of microorganisms commonly encountered in bioprocessing industries. Since these microorganisms mutually differ from each other regarding morphology, size, and attraction towards different material.

The mixture of test microorganisms is then applied 102 at at least one position in the bioprocessing equipment.—The test microorganisms are allowed to sediment, attach to surfaces of the equipment and form a biofilm during a waiting period 103 of at least 2 hours. Thereafter, the bioprocessing equipment is sanitized 104 using for example sodium hydroxide, isopropanol, ethanol, peracetic acid, and/or hydrogen peroxide. The agent(s) used for sanitization may be left in the bioprocessing equipment for about 1 hour, i.e. the hold-time may be about 1 hour.

A test sample, a liquid volume, a contact plate or a swab or tops test, is then collected 104 from at least one point in the bioprocessing equipment after the sanitization step, and possibly also 105 after application and/or immediately before the sanitization step. (In some case, the bioprocessing equipment may have to be disassembled in order for test samples to be taken at specific positions in the equipment, such as at in flow paths etc.)

If the test sample is a liquid volume, or if transferred to a liquid volume, the liquid volume may be filtered 110 through filters with a suitable pour size (for example: 0.45 μm) to capture presence of organisms in the filter material or be filtered such that microorganisms are collected in the filtrate.

The collected test sample or samples are each transferred 107 into/onto the same culture medium that promotes growth and colony formation of all microorganism strains in the mixture of microorganisms applied in the bioprocessing equipment such that strains are individually distinguishable from each other. Test samples may be serially diluted in e.g. sodium chloride before being transferred into/onto suitable culture media. The culture media is incubated until individual CFU: s can be counted and from that concentrations of challenging microorganism strains in the original test samples estimated.

The culture medium may be a broth or may be a solid or semi-solid culture medium (i.e. containing agar at a concentration of 1.5 to 2.0 % concentration or agar at a concentration of 0.2-0.5 %, respectively). The broth/agar used may for example be Luria-Bertani (LB) broth, Tryptic Soy Broth (TSB), Tryptic soy agar (TSA), nutrient broth, Sabouraud dextrose broth, Brain Heart Infusion (BHI) broth, Mueller-Hinton broth, MacConkey broth, cetrimide broth, chocolate agar, Columbia broth, mannitol salt broth, and blood broth.

Incubation of the culture medium, allowing possibly present microorganisms to grow and differentiate, may be performed at a temperature of 20-42 °C, during a time-period of 1 to 5 days, at a pH of 6.0-8.0 and an osmotic pressure of 0.05-2.5 M.

The osmotic range may have an optimal range between 0.05-0.5 M (i.e. NaCl). A optimal pH range may be between 6.5-7.5. An optimal temperature range may be between 30-37 °C. The incubation may take place at atmospheric pressure (101.3 kPa).

The culture medium may comprise a carbon source selected from any one or more of glucose (1-2%), lactose (0.5-2%), sucrose (0.5-1%), glycerol (0.1-1%), fructose (1-2%), mannitol (0.5-2%), succinate (0.1-1%), maltose (0.5-1%), xylose (0.5-1%), galactose(1%-2% and other simple sugars and organic acids.

The culture medium may comprise a nitrogen source selected from any one or more of peptone: 5-20 g/L, tryptone: 5-20 g/L, yeast extract: 1-10 g/L, ammonium sulfate: 0.5-2 g/L, ammonium chloride: 0.5-2 g/L.

The culture medium may comprise any one or more of trace elements and co-factors: iron: 1-10 mg/L, manganese: 0.1-1 mg/L, zinc: 0.1-1 mg/L, copper: 0.1-1 mg/L, cobalt: 0.1-1 mg/L, molybdenum: 0.1-1 mg/L, nickel: 0.1-1 mg/L, selenium: 0.1-1 mg/L, biotin: 0.1-1 μg/L, folic acid: 0.1-1 μg/L, nicotinic acid: 1-10 μg/L, pantothenic acid: 0.1-1 μg/L, pyridoxine: 0.1-1 μg/L, riboflavin: 0.1-1 μg/L, thiamine: 0.1-1 μg/L, vitamin B 12: 0.01-0.1 μg/L, vitamin K: 0.1-10 μg/L, inositol: 1-100 mg/L, PABA (para-aminobenzoic acid): 1-10 mg/L.

The mixture of microorganisms can be grown in for example the following buffers: phosphate buffer, MOPS (3-(N-morpholino)propane sulfonic acid) buffer, HEPES (4-(2-hydroxyethyl)-1-piperazineethanesulfonic acid) buffer, tris (tris(hydroxymethyl)aminomethane) buffer.

All above mentioned organisms can be grown in a shaking or stirred liquid culture, broth, with aeration rates of approximately 150-250 rpm and a headspace-to-medium ratio of 1:2 to 1:10.

Example of isotonic solutions that can be used when culturing the microorganisms in/on the culture medium are: phosphate-buffered saline (PBS), (10 mM-100 mM, pH 7.2-7.4), sodium chloride solution (50 mM-1 M), tris-EDTA (TE) buffer (10 mM-100 mM, EDTA 0.1 mM-10 mM, pH 8.0), MOPS (3-(N-Morpholino) propane sulfonic acid) buffer (10 mM-50 mM, pH 7.0-7.4), HEPES (4-(2-Hydroxyethyl)-1-piperazineethane sulfonic acid) buffer (10 mM-50 mM, 7.0-8.0), potassium phosphate buffer (10 mM-100 mM, pH 7.0-7.4), sodium acetate buffer (10 mM-100 mM, pH 4.5-5.5), tris-HCl buffer (10 mM-100 mM, pH 7.4) During incubation 107 of the culture medium, possibly present microorganisms are allowed to grow and differentiate. Microorganisms are identified and/or the amount of microorganisms of each microorganism strain grown on/in the culture medium counted 108. In broth, motile bacteria will create a uniform cloudiness in the broth. Non-motile bacteria with waxy cell walls tend to float on the surface of the broth. Other types of non-motile bacteria may create sediment at the bottom of the broth.

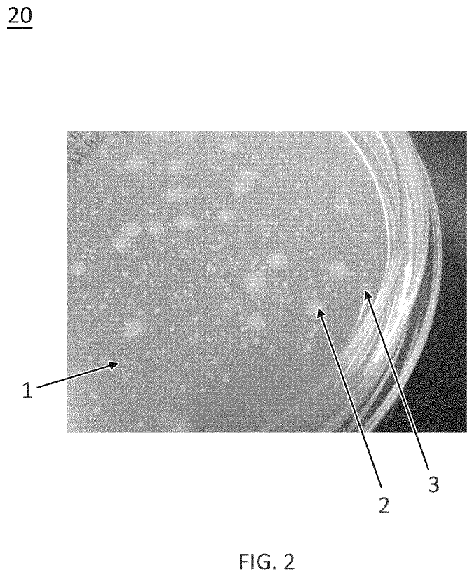

In FIG. 2 is shown an agar plate 20 with three co-cultured microorganisms: 1) Staphylococcus aureus (ATCC 6538), 2) Escherichia coli (ATCC 8739) and 3) Pseudomonas aeruginosa (ATCC 9027).

Traditionally, a sanitization procedure may be evaluated by challenging the system with one test microorganism at a time, which is then evaluated. If additional microorganisms are used, these are tested in sequence. The more test microorganisms, the longer the evaluation time. Such sanitization studies are, therefore, both time consuming and costly.

The above described procedure, as well as the traditional procedure, give information on how the hygienic design performs and/or how used sanitization procedure performs and whether a change in the hygienic design and/or sanitization process is needed or not. Presence of one or more test microorganism, and possibly also other organisms present in the system, and amounts thereof in/on the culture medium is an indication that hygienic design and/or the sanitization procedure need to be changed.

Compared to the traditional procedure, the present procedure uses two or more test microorganisms mixed together before application. Thereby, there is a significant reduction in the time, possibly from three/four months into one month, compared to if microorganisms are sequentially evaluated.

The mixture of test microorganism strains used may be prepared by preparing separate culture plates with suitable culture medium and one challenging microorganism strain on each plate. The plates are incubated until distinct colonies have been formed. Single colonies from each microorganism strain are then transferred into separate suitable culture media, incubated at 32-37 °C during shaking until suitable optical densities (ODs) are obtained. Incubated organisms are then diluted and mixed in a solution that provides a steady-state environment for the microorganism strains at a pre-defined concentration, such as >106 CFU/mL. Example of a steady-state solution is 0.05-1 M sodium chloride. The prepared solution with mixed test microorganisms is applied at one or more positions in the bioprocessing equipment using suitable and realistic parameters. If the bioprocessing equipment is a chromatography system, such realistic parameters may include flow speed, back pressure, and time.

After sanitization, the sanitization agent may be replaced with a suitable neutralization solution until no traceable amount of sanitization agent is found. Example of neutralization solution: 0.2 M sodium acetate, 20% ethanol, pH 5.5.

### Experimental Example

Parts of a bioprocessing equipment comprising hydrophobic/inert material was used. The parts comprised several different test sample collection sites and was evaluated based on hygienic design and sanitization procedure used. Studies were conducted using 1 M NaOH as sanitization agent, contact time ≥1 h. The parts were challenged with Escherichia coli (Ec), Pseudomonas aeruginosa (Pa) and Staphylococcus aureus (Sa) mixed into a suspension with a concentration >10⁶ CFU/mL of each organism.

### Pre-Cleaning

The bioprocessing equipment parts were pre-cleaned with a detergent and small brushes. Ethanol (70%) was sprayed on the cleaned surfaces and left to be air dried. In study 3, the ethanol was exchange for a sporicidal agent called Prochlor. This agent was applied using the same procedure.

### Preparation and Application of Challenging Test Microorganism Suspension

Tryptic soy agar (TSA) plates streaked with the challenging test microorganisms were incubated in 37 °C overnight (O.N.). Fresh colonies from those plates were transferred to 200 mL of autoclaved Bacto Tryptic Soy Broth (TSB) media and left shaking in 37 °C O.N. Based on measured ODs of the pre-cultures and the assumption that 1 OD is approximately 10⁹ CFU/mL for Ec, 2×10⁹ CFU/mL for Pa and 1.5×10⁸ CFU/mL for Sa, calculations were made on the volumes needed of the pre-cultures to be added in 25-55 L of filtered 0.9% NaCl solution to obtain the final concentrations of ~10⁷ CFU/mL for each challenging test microorganism in the suspension.

The suspension was applied on the parts of the bioprocessing equipment at multiple sites. The infected system was left for 16-20 h in room temperature (RT) before starting the sanitization procedure.

### Sanitization Procedure

Liquids were filtered through 0.2 μm filters except for the sanitization agent 1 M NaOH. The procedure was the same as when pre-cleaning.

Before microbial sampling liquid was removed from the parts of the bioprocessing equipment.

### Microbial Sampling

Microbial samples were taken at predetermined sites of the parts of the bioprocessing equipment.

Test method 1, microbial air sampling: Sampling of air for airborne microorganisms was conducted with a Microbial Air Sampler (MAS). A MAS loaded with an agar plate was positioned at a suitable measuring point. When the measuring starts, a pre-defined volume of surrounding air is passed through the machine. Microorganisms will be collected on the agar surface by impaction.

Test method 2, direct filtration: Sample solutions (minimum 10 mL) were collected in sterile tubes and then filtered through a 0.45 μm cellulose nitrate membrane filters. Filters were incubated on agar plates at 30-35 °C for 5 days after which the plates were inspected for CFUs.

Test method 3, swab: Surface samples were taken with swabs. The swab was inserted into the tube containing the isotonic swab rinse solution and vortexed for a minimum of 20 s. The solutions including the swabs were poured into Petri dishes and mixed with 30 mL of temperature controlled molten agar. Maximum temperature of the molten agar should be 45 °C. After solidification, plates were incubated at 30-35 °C for 5 days after which the plates were inspected for CFUs.

Samples of challenging test organism suspensions were diluted in series in 0.9% NaCl. Samples from the diluted suspensions were plated on agar plates and incubated at 30-35 °C for 1-2 days after which the plates were inspected for CFU: s. The concentration of challenging test organism was determined in the sampled suspensions.

### Results

First, a sanitization procedure with 1 M NaOH and a contact-time of 1 hour was used. Two out of three challenging organisms (Pseudomonas aeruginosa and Escherichia coli) were eradicated, meanwhile the third (Staphylococcus aureus) was found in some sample points of the parts of the bioprocessing equipment. Contaminants other than the challenging test organisms: Gram-positive cocci, Gram-positive rod (spore former) and Bacillus cereus/thuringiensis/mycoides (spore former) were also found at some horizontal test points. Spores are in general more difficult to eradicate than vegetative microbes. The design and position of some parts may trap liquid, which could be a potential risk for contamination and biofilm build up.

This sanitization procedure did not eradicate the spores found in the horizontal portions. Biofilm can also be a possible factor that adds to the resistance against the sanitization agent.

Staphylococcus aureus is a gram-positive cocci with the size ~1 μm, which is smaller than the other two rod shaped organisms (1-5 μm in length and 0.5-1 μm in diameter). Those cocci can probably reach and hide in pockets where the other two challenging microorganisms cannot reach. Therefore, Staphylococcus aureus is more difficult to eradicate than the other two challenging test microorganisms.

The results indicated that the contact time should be increased and the sanitization process should be modified to improve the sanitization. The problem with the horizontal portions mentioned indicates that the hygienic design used is not optimal and that problems with biofilm may occur.

Various aspects and embodiments of the present invention thus provide an improved method for evaluating sanitization procedures. The method entails adding multiple different organisms and aims to keep them alive and measurable all the way up to a point where they are then subsequently sanitized (as such the method is not of itself merely just a sanitization method where the objective is to remove organisms per se). Such a method may thus enable the use of several different organisms at the same time, whilst nevertheless being able to identify/quantify each of them separately as they grow together on the same growth media (e.g. on a shared agar plate) during the whole evaluation procedure.

1. A procedure for evaluating a sanitization procedure used for sanitizing bioprocessing equipment or for evaluating the hygienic design of bioprocessing equipment, comprising:
pre-cleaning the bioprocessing equipment,
providing a mixture of test microorganisms comprising two or more different microorganism strains,
applying the mixture of test microorganisms in the bioprocessing equipment,
waiting for at least 2 hours,
sanitizing the bioprocessing equipment,
collecting at least one test sample, possibly comprising test microorganisms, from at least one point in the bioprocessing equipment after the sanitization,
transferring at least a portion of a collected test sample into/onto a culture medium,
incubating the culture medium, allowing possibly present test microorganisms to grow and differentiate in/on the culture medium,
identifying and/or counting the amount of test microorganisms of each microorganism strain grown on/in the culture medium.

2. The procedure of claim 1, wherein the mixture of test microorganisms comprises two or more different test microorganism strains selected from Escherichia coli, Pseudomonas aeruginosa, Staphylococcus aureus, Candida albicans, Bacillus subtilis, Aspergillus niger, Pichia pastoris and Saccharomyces cerevisiae.

3. The procedure of claim 2, wherein the mixture of test microorganisms comprises 2-4 strains of different microorganisms.

4. The procedure of claim 1, wherein the step of waiting for at least 2 hours is performed at a temperature of 15-30 °C.

5 The procedure of claim 1, wherein sanitization of the bioprocessing equipment is made using any one or more of sodium hydroxide, isopropanol, ethanol, peracetic acid, hydrogen peroxide, acetic acid, citric acid, sodium hypochlorite, quaternary ammonium compounds, iodine-based disinfectants, phenols, aldehydes, ammonia, nitiric acid, phosphoric acid, sulfuric acid, peroxyacetic acid, potassium permanganate, potassium hydroxide, ammonium chloride, sodium bicarbonate, chlorhexidine and perethylene.

6. The procedure of claim 1, wherein a test sample further is collected from at least one point in the bioprocessing equipment after application and/or immediately before the sanitization, each test sample being transferred into/onto a culture medium, incubated to allow possibly present test microorganisms to grow and differentiate, and microorganism strains grown on/in the culture medium are identified and/or the number of microorganisms counted

7. The procedure of claim 1, comprising a step of filtering a collected test sample before transferring at least a portion of the filtered sample or at least a portion of the filter into/onto a culture medium.

8. The procedure of claim 1, wherein incubation of the culture medium, allowing possibly present microorganisms to grow and differentiate, is made at a temperature of 20-42 °C, during a time-period of 1 to 5 days, at a pH of 6.0-8.0 and at an osmotic pressure of 0.05-2.5 M.

9. The procedure of claim 1, wherein the culture medium is a solid or semi-solid culture medium.

10. The procedure of claim 1, wherein the culture medium is a broth.

11. The procedure of claim 1, wherein the culture medium comprises a carbon source selected from any one or more of glucose, lactose, sucrose, glycerol, fructose, mannitol, succinate, maltose, xylose, and galactose.

12. The procedure of claim 1, wherein the culture medium comprises a nitrogen source selected from any one or more of peptone, tryptone, yeast extract, ammonium sulfate, and ammonium chloride.

13. procedure of claim 1, wherein the culture medium comprises any one or more of iron, manganese, coper, cobalt, molybdenum, nickel, selenium, biotin, folic acid, nicotinic acid, pantothenic acid, pyridoxine, riboflavin, thiamine, vitamin B12, vitamin K, inositol and para-aminobenzoic acid.
